(19) United States
(12) Patent Application Publication
(10) Pub. No.: US 2013/0012747 A1
(43) Pub. Date: Jan. 10, 2013

(54) METHOD OF MANUFACTURE OF AROMATIC COMPOUND

(75) Inventors: Hongtao Ma, Shinagawa-ku (JP); Yo Yamamoto, Numazu-shi (JP); Yuji Ogawa, Kawagoe-shi (JP)

(73) Assignee: Meidensha Corporation, Tokyo (JP)

(21) Appl. No.: 13/636,257

(22) PCT Filed: Feb. 10, 2011
(86) PCT No.: PCT/JP2011/052809

§ 371 Date: Sep. 20, 2012

(30) Foreign Application Priority Data
Mar. 23, 2010 (JP) 2010-066077

(51) Int. Cl. C07C 2/76; C07C 5/41

(52) U.S. Cl. 585/417; 585/418

(57) ABSTRACT

In manufacturing aromatic hydrocarbons by causing a contact reaction between a lower hydrocarbon and a catalyst, the aromatic hydrocarbons are stably produced over a long period of time while maintaining high aromatic hydrocarbon yields. The process includes a reaction process of initiating the contact reaction between the lower hydrocarbon and the catalyst thereby obtaining the aromatic hydrocarbons and hydrogen, and a regeneration process of regenerating the catalytic activity by bringing hydrogen into contact with the catalyst used in the reaction process. The reaction process and the regeneration process are repeated thereby producing the aromatic hydrocarbons and hydrogen. In the reaction process, carbon monoxide is added to the lower hydrocarbons and additionally a reaction temperature is set at higher than 800° C.

### TECHNICAL FIELD

The present invention relates to advanced uses of gases which contain methane as a principal component, such as natural gas, biogas and methane hydrate. The present invention particularly relates to a chemical catalytic conversion technique for producing aromatic compounds (containing benzene and naphthalene as principal components, which are materials for chemical products such as plastics and the like) and a high purity hydrogen gas from methane.

### BACKGROUND OF THE INVENTION

Natural gas, biogas and methane hydrate are regarded as the most effective energy resources against global warming, and therefore an interest in techniques using them has been growing. A methane resource is expected to be a novel organic resource in the next generation and to be a hydrogen resource for use in fuel cells, by virtue of its clean property.

As a method for manufacturing aromatic compounds such as benzene and the like and hydrogen from methane, a process for reacting methane in the presence of a catalyst has been known, as discussed in Non-Patent Publication 1. As the catalyst used in this process, molybdenum impregnated on ZSM-5 is said to be an effective one.

However, there are problems of serious carbon formation and low methane conversion rate even in the case of using such a catalyst. Carbon formation in particular is a serious problem directly associating with a degradation phenomenon of the catalyst.

In order to solve these problems, Patent Publication 1 discloses that a mixture gas obtained by adding CO₂ or CO to methane is provided to a catalytic reaction under a condition where the temperature for the catalytic reaction is set within a range of from 300 to 800° C. With the addition of CO₂ or CO, carbon formation is inhibited and additionally catalyst degradation is prevented, thereby allowing stable production of aromatic compounds.

In Patent Publications 2 and 3, a reaction for producing aromatic compounds and a reaction for regenerating a catalyst used in the aromatic compound-producing reaction are alternately switched thereby to inhibit the catalyst from degradation with time so as to maintain the catalytic reaction. In other words, a lower hydrocarbon serving as a substrate for the reaction, and a hydrogen-containing gas (or a hydrogen gas) for maintaining or regenerating the catalyst are periodically alternately brought into contact with the catalyst.

### REFERENCES ABOUT PRIOR ART

### Patent Publication

Patent Publication 1: Japanese Patent Provisional Publication No. 11-060514

Patent Publication 2: Japanese Patent Provisional Publication No. 2003-026613

Patent Publication 3: Japanese Patent Provisional Publication No. 2008-266244

### Non-Patent Publication

Non-Patent Publication 1: “JOURNAL OF CATALYSIS” 1997, volume 165, pages 150-161

### SUMMARY OF THE INVENTION

### Problems to be Solved by the Invention

Of the problems as discussed with citing the above conventional techniques, the catalyst degradation caused by the carbon formation and exemplified by Non-Patent Publication 1 is critically important for stably producing aromatic hydrocarbons and the like over a long period of time in a fixed-bed reaction system in particular.

In view of this, Patent Publication 1 proposes a process for initiating a contact reaction between a feedstock gas and a catalyst with the addition of CO₂ or CO on the condition that the reaction temperature ranges from 300 to 800° C., thereby inhibiting the carbon formation so as to prevent the catalyst degradation. According to this process, the catalyst is greatly improved in stability but nevertheless tends to be reduced in maximal benzene yield.

Meanwhile, there is proposed in Patent Publication 2 a process in which a deposition of hard-to-remove cokes is prevented by switching between a reaction gas and a hydrogen gas or hydrogen-containing gas at certain intervals thereby obtaining aromatic compounds stably over a long period of time. In this process, a regeneration treatment is performed before the deposited carbon accumulates, in which it is possible to maintain the benzene yield (which serves as an index of a catalytic activity) over a long period of time. Incidentally, the benzene yield depends on that in an initial stage of the reaction.

In the initial stage of the reaction, hydrocarbons converted from methane are to convert into benzene with high probabilities due to the catalytic action, since the amount of the deposited carbon is small. With increasing a methane-conversion ratio (for example, by setting the reaction temperature at 800° C. or more), it becomes possible to obtain a higher benzene yield in the initial stage of the reaction. However, in the case of increasing the methane-conversion ratio by performing the reaction at high temperatures, there comes up a problem where the carbon deposition becomes remarkable and the catalyst degradation is accelerated by the accumulation of carbon.

Hence there is strongly desired a process which effectively acts on removal of the deposited carbon even at high temperatures and which never reduces the maximal benzene yield.

### Means for Solving the Problems

The method for manufacturing aromatic hydrocarbons according to the present invention, which can solve the above-mentioned problems, is a method for producing hydrogen and an aromatic compound containing an aromatic hydrocarbon as the principal component by initiating a contact reaction between a lower hydrocarbon and a catalyst, characterized in that: carbon monoxide is added to the lower hydrocarbon; and a reaction temperature is higher than 800° C.

In the method for manufacturing aromatic hydrocarbons, it is preferable that the carbon monoxide has a concentration of from 0.75 to 20% relative to a reaction gas. In the method for manufacturing aromatic hydrocarbons, it is further preferable that the reaction temperature is not lower than 820° C.

In the method for manufacturing aromatic hydrocarbons, it is still further preferable that the aromatic hydrocarbon is produced by repeating a reaction process of initiating the contact reaction between the lower hydrocarbon and the catalyst and a regeneration process of regenerating the catalyst used in the reaction process.

### Effects of the Invention

According to the present invention, it is possible to contribute to inhibition on the catalyst degradation and to improvement in aromatic compound yield, at the time of producing aromatic compounds by inducing the contact reaction between a lower hydrocarbon and a catalyst.

### BRIEF DESCRIPTION OF THE DRAWING

FIG. 1 is a graph showing changes in benzene yield with time, obtained by continuously conducting a catalytic reaction in the presence of a Mo-HZSM5 catalyst;

FIG. 2 is a graph showing changes in benzene yield with time, obtained in the case of continuously conducting a catalytic reaction in the presence of a Mo-HZSM5 catalyst (with the addition of CO);

FIG. 3A is a graph showing changes in benzene yield with time, obtained in the case of repeating a catalytic reaction process and a catalyst regeneration process;

FIG. 3B is a graph showing changes in benzene formation rate with time, obtained in the case of repeating the catalytic reaction process and the catalyst regeneration process;

FIG. 3C is a graph showing changes in methane conversion ratio with time, obtained in the case of repeating a catalytic reaction process and a catalyst regeneration process;

FIG. 4 is a graph showing changes in benzene amount in 100 μl of gas with time, obtained after the reaction in the case of adding carbon monoxide; and

FIG. 5 is a graph showing changes in benzene amount in 100 μl of gas with time, obtained after the reaction in the case of not adding carbon monoxide.

### DETAILED DESCRIPTION OF THE INVENTION

The present invention is an invention relating to a method for manufacturing aromatic compounds (containing benzene and naphthalene as principal components) and a high purity hydrogen gas, by initiating a contact reaction between a lower hydrocarbon and a catalyst for converting lower hydrocarbons into aromatic compounds (which catalyst is hereinafter referred to as merely “a catalyst”). The present invention is characterized in that the contact reaction is conducted with the addition of carbon monoxide to a reaction gas served to the contact reaction at a temperature of higher than 800° C.

According to the present invention which relates to the method for manufacturing aromatic compounds, it is possible not only to inhibit degradation of the catalytic activity of the catalyst used in the reaction but also to improve the maximal benzene yield more greatly than in a contact reaction made between pure methane and the catalyst.

Additionally, to perform a catalytic reaction process and a catalyst regeneration process alternately allows keeping the reaction stable over a long period of time while maintaining a high yield without accumulating hard-to-remove cokes.

An embodiment of the catalyst used for the method for manufacturing aromatic compounds according to the present invention is, for example, a metallosilicate on which a catalytic metal is carried.

Examples of the metallosilicate on which the catalytic metal is carried include a molecular sieve 5A which is a porous material formed of silica and alumina, faujasite (NaY and NaX), ZSM-5 and MCM-22, in the case where the metallosilicate is an aluminosilicate, for example. Furthermore, there are also included, for instance: a zeolite substrate characterized by being a porous material containing phosphoric acid as a principal component and by having micropores and channels of 6-13 angstroms, such as ALPO-5, VPI-5 and the like; and a mesoporous substrate characterized by containing silica as a principal component and alumina as one component and by having cylindrical pores (or channels) of mezopores (10-1000 angstroms), such as FSM-16, MCM-41 and the like. In addition to these aluminosilicates, metallosilicates formed of silica and titania, and the like can be also used as the catalyst.

Moreover, it is preferable that the metallosilicate used in the present invention has a surface area of from 200 to 1000 m²/g and has micropores or mesopores of within a range of from 5 to 100 angstroms. When the metallosilicate is an aluminosilicate, for example, it is possible to use one having a content ratio between silica and alumina (silica/alumina) of from 1 to 8000 as generally available porous materials; however, it is further preferable to set the content ratio (silica/alumina) within a range of from 10 to 100 in order to perform the aromatization reaction of lower hydrocarbons of the present invention at a practical lower hydrocarbon conversion rate and at a practical aromatic compound selectivity.

As the metallosilicate, those of a proton exchange type (a type H) are normally used. Additionally, a part of protons thereof may be exchanged for one kind of cations selected from the group consisting of alkali metals such as Na, K, Li and the like, alkali-earth metal elements such as Mg, Ca, Sr and the like, and transition metal elements such as Fe, Co, Ni, Zn, Ru, Pd, Pt, Zr, Ti and the like. Furthermore, the metallosilicate may contain Ti, Zr, Hf, Cr, Mo, W, Th, Cu, Ag and the like in a right amount.

Moreover, it is preferable to use molybdenum as the catalytic metal of the present invention, in which rhenium, tungsten, iron, cobalt also may be acceptable. These catalytic metals may be carried on the metallosilicate in combination. Furthermore, one kind of element selected from the group consisting of the alkali-earth metals such as Mg and the like and the transition metal elements such as Ni, Zn, Ru, Pd, Pt, Zr, Ti and the like may be carried on the metallosilicate together with the above-mentioned catalytic metal.

In the case of carrying the catalytic metal (or a precursor containing the catalytic metal) on the metallosilicate, the weight percentage of the catalytic metal to the substrate is within a range of from 0.001 to 50%, preferably within a range of from 0.01 to 40%. As a method for carrying the catalytic metal on the metallosilicate, there has been known a method of conducting a heat treatment under an atmosphere of inert gas or oxygen gas after carrying the catalytic metal on a metallosilicate substrate by impregnating the substrate with an aqueous solution or an organic solvent solution (such as alcohol and the like) containing the precursor of the catalytic metal or by ion-exchange process. Examples of the precursor containing molybdenum which is one of the catalytic metals include halide such as chloride, bromide and the like, mineral oxides such as nitrate, sulfate, phosphate and the like, and carboxylate such as carbonate, acetate, oxalate and the like, in addition to ammonium paramolybdate, ammonium phosphomolybdate and a 12-type molybdic acid.

Referring now to an example case of using molybdenum as the catalytic metal, the method for carrying the catalytic metal on the metallosilicate will be discussed. First of all, a metallosilicate substrate is impregnated with an aqueous solution of ammonium molybdate thereby carrying the catalytic metal thereon. Then, a solvent is removed from the substrate by drying under reduced pressure. A heat treatment is thereafter conducted in a nitrogen-containing oxygen flow or pure oxygen flow at a temperature of from 250 to 800° C. (preferably from 350 to 600° C.), thereby producing a molybdenum-carried metallosilicate catalyst.

The metallosilicate catalyst on which the catalytic metal is carried is not particularly limited to the above-mentioned embodiment and therefore may have any form such as powder, granules and the like. Additionally, the metallosilicate catalyst on which the catalytic metal is carried may be formed in pellets or an extrusion with the addition of a binder such as silica, alumina, clay and the like.

In the present invention, “lower hydrocarbon(s)” refers to methane or saturated or unsaturated hydrocarbons having 2 to 6 carbon atoms. Examples of the saturated or unsaturated hydrocarbons having 2 to 6 carbon atoms are ethane, ethylene, propane, propylene, n-butane, isobutane, n-butene, isobutene and the like.

A reactor used in the method for manufacturing aromatic compounds according to the present invention may be a fixed-bed reactor, a fluidized-bed reactor or the like. Referring now to some embodiments, the method for manufacturing aromatic compounds according to the present invention will be discussed in detail.

(1) Changes in Catalytic Activity with the Addition of Carbon Monoxide

### Referential Example 1

In Referential Example 1, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 0.75% carbon monoxide relative to a reaction gas at a temperature of 780° C.

As a metallosilicate substrate of Referential Example 1 of the present invention, a catalyst was produced in the use of an H-type ZSM5 zeolite (SiO₂/Al₂O₃=40) by the following preparation method.

400 g of HZSM5 was added to an aqueous solution obtained by dissolving 456.5 g of ammonium molybdate in 2000 ml of ion-exchange water, followed by stirring at room temperature for 3 hours, thereby carrying molybdenum on HZSM5 by impregnation.

After drying the thus obtained molybdenum-carried HZSM5 (Mo-HZSM5), the substrate was calcined under atmospheric conditions at a temperature of 550° C. for 8 hours, thereby obtaining a catalyst powder on which molybdenum is carried (i.e., a catalyst having a weight percentage of molybdenum of 6.8 wt % relative to the total of the catalyst). Furthermore, the catalyst powder is extruded into in a pellet form upon the addition of an inorganic binder, and then calcined thereby preparing a catalyst.

The catalyst prepared by the above-mentioned method was charged into a reaction column (18 mm internal diameter) of a fixed-bed flow reactor, the reaction column being formed of Inconel 800H whose portion to be brought into contact with gas is subjected to a calorizing treatment. Upon setting the reaction temperature and the pressure of the interior of the reaction column at 780° C. and 0.3 MPa, respectively, methane to which 0.75% carbon monoxide was added relative to the reaction gas was supplied thereinto at a flow rate or a space velocity (SV) of 3000 ml/hr/g-MFI, thereby conducting an aromatization reaction of methane. Then, an evaluation of the catalytic activity in a reaction for forming benzene and hydrogen by the aromatization reaction of methane was performed.

The catalytic activity was evaluated on the basis of “benzene yield”, “methane conversion ratio” and “benzene formation rate”. Incidentally, “benzene yield”, “methane conversion ratio” and “benzene formation rate” in Examples were defined as follows:

“Benzene yield (%)”=[“an amount of a formed benzene (mol)”/“an amount of methane provided to a methane-reforming reaction (mol)”]×100

“Methane conversion ratio (%)”=[(“a flow rate of a feedstock methane”−“a flow rate of an unreacted methane”)/“the flow rate of the feedstock methane”]×100

“Benzene formation rate (nmol/g/s)”=“the number of nanomoles (nmol) of benzene produced on 1 g of the catalyst per second”

As a pretreatment for the catalyst conducted before feeding the reaction gas, the catalyst was increased in temperature up to 550° C. in air flow and kept therein for 2 hours. Then, the air flow was switched to a pretreatment gas containing 20% methane and 80% hydrogen and the temperature was increased to 700° C. and kept thereat for 3 hours. The pretreatment gas was then switched to the reaction gas and the temperature was increased up to a certain degree (780° C.), followed by carrying out an evaluation of the catalyst.

Hydrogen, argon, methane were analyzed by TCD-GC, while aromatic hydrocarbons such as benzene, toluene, xylene, naphthalene and the like were analyzed by FID-GC.

### Referential Example 2

In Referential Example 2, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 6.4% carbon monoxide relative to a reaction gas at a temperature of 780° C.

Since the catalyst used in the method for manufacturing aromatic compounds according to Referential Example 2 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

### Comparative Example 1

In Comparative Example 1, the method for manufacturing aromatic compounds according to the present invention was embodied by initiating a contact reaction between methane only and a catalyst in such a manner as to initiate a catalytic reaction at a temperature of 780° C. thereby producing an aromatic compound and hydrogen gas.

Since the catalyst used in the method for manufacturing aromatic compounds according to Comparative Example 1 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of a gas supplied to the reaction were all the same to those in Referential Example 1, so that a detailed explanation therefor is omitted too.

### Comparative Example 2

In Comparative Example 2, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 1.2% carbon dioxide relative to a reaction gas at a temperature of 780° C.

Since the catalyst used in the method for manufacturing aromatic compounds according to Comparative Example 2 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

### Comparative Example 3

In Comparative Example 3, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 3.0% carbon dioxide relative to a reaction gas at a temperature of 780° C.

Since the catalyst used in the method for manufacturing aromatic compounds according to Comparative Example 3 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

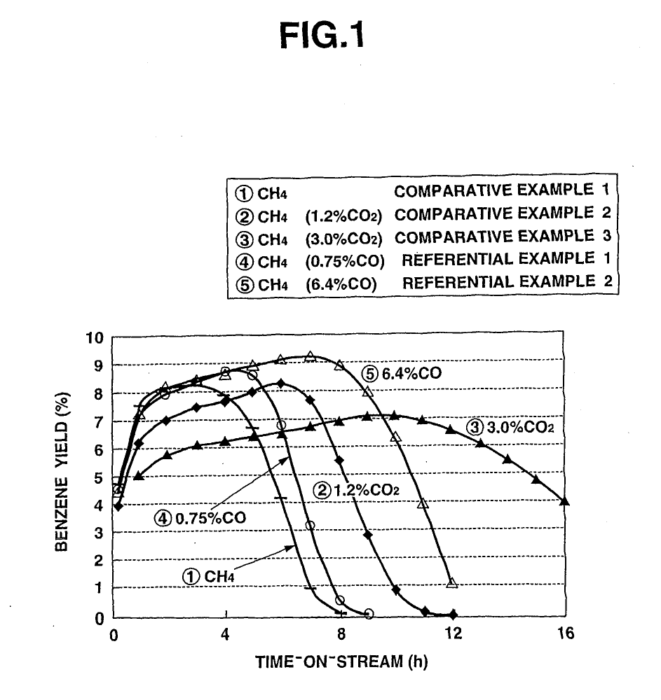

FIG. 1 is a graph showing changes in benzene yield with time, obtained by continuously conducting a catalytic reaction in the presence of a Mo-HZSM5 catalyst under reaction conditions discussed in Referential Examples 1 and 2 and Comparative Examples 1 to 3.

It is found therefrom that the catalyst activity is lost after 7 hours of reaction in the case of using pure methane during the contact reaction (Comparative Example 1) while that in the case of using a carbon dioxide-added methane during the contact reaction (Comparative Examples 2 and 3) is inhibited from reduction. Particularly with the addition of 3.0% carbon dioxide (Comparative Example 3), the early maximal benzene yield is maintained even after 15 hours of reaction.

As compared with the case where pure methane was used during the contact reaction (Comparative Example 1) thereby exhibiting a maximal benzene yield of not lower than 8.0%, the case where carbon dioxide was added (for example, Comparative Example 3) exhibits a maximal benzene yield of 7.0% and therefore inferior in maximal benzene yield. Additionally, it is apparent from comparison between Comparative Examples 2 and 3 that the maximal benzene yield decreases with increase of the amount of added carbon dioxide.

This means that the addition of carbon dioxide to the reaction gas increases the time during which the catalyst can keep its activity but decreases the maximal benzene yield if the reaction temperature is constant.

On the other hand, the case of initiating the contact reaction between the catalyst and the carbon monoxide-added methane (Referential Example 1) is improved in maximal benzene yield as compared with the case of initiating the contact reaction between pure methane and the catalyst (Comparative Example 1). Particularly from comparison between Referential Examples 1 and 2, it is apparent that an increase in the added amount of carbon monoxide relative to the reaction gas improves not only the stability of the catalytic activity but also the maximal benzene yield.

(2) Changes in Catalytic Activity, Caused by the Difference in Added Amount of Carbon Monoxide

### Referential Example 3

In Referential Example 3, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 1.5% carbon monoxide relative to a reaction gas at a temperature of 780° C.

Since the catalyst used in the method for manufacturing aromatic compounds according to Referential Example 3 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

### Referential Example 4

In Referential Example 4, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 3.0% carbon monoxide relative to a reaction gas at a temperature of 780° C.

Since the catalyst used in the method for manufacturing aromatic compounds according to Referential Example 4 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

### Referential Example 5

In Referential Example 5, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 11.9% carbon monoxide relative to a reaction gas at a temperature of 780° C.

Since the catalyst used in the method for manufacturing aromatic compounds according to Referential Example 5 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

### Referential Example 6

In Referential Example 6, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 20% carbon monoxide relative to a reaction gas at a temperature of 780° C.

Since the catalyst used in the method for manufacturing aromatic compounds according to Referential Example 6 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Referential Example 1, so that a detailed explanation therefor is omitted too.

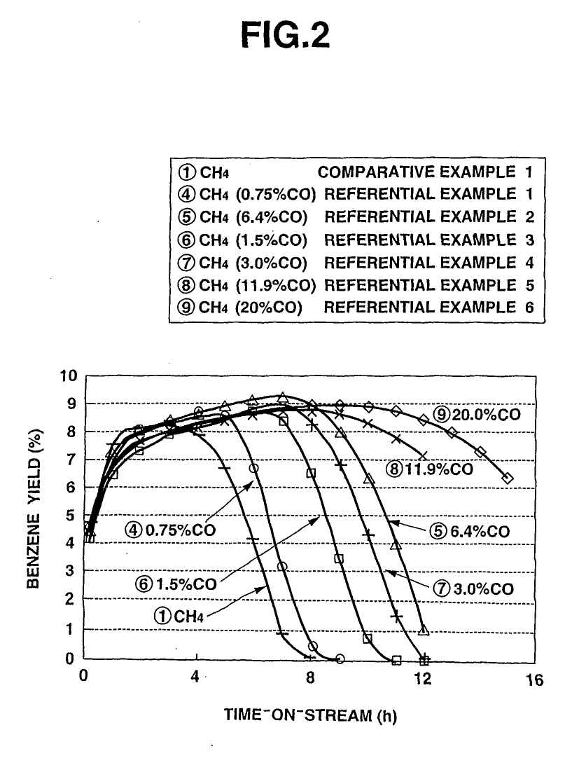

FIG. 2 is a graph showing changes in benzene yield with time, obtained by continuously conducting a catalytic reaction in the presence of a Mo-HZSM5 catalyst under reaction conditions discussed in Referential Examples 1 to 6 and Comparative Example 1.

The case where the carbon monoxide-added methane was used in the contact reaction (Referential Examples 1 to 6) was not only prevented from degradation in catalytic activity but also improved in maximal benzene yield as compared with the case where pure methane was used in the contact reaction (Comparative Example 1), not depending on the added amount of carbon monoxide. It is apparent that an increase in carbon monoxide improves the effect of inhibiting the degradation of the catalytic activity. The maximal benzene yield was little impaired even if the added amount of carbon monoxide was as much as 20%, indeed the maximal benzene yield exceeded 9% with the addition of 6.4% carbon monoxide.

(3) Changes in Catalytic Activity in the Case of Repeating a Catalytic Reaction Process and a Catalyst Regeneration Process, Caused by the Difference in Added Amount of Carbon Monoxide

### Example 1

In Example 1, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 3.0% carbon monoxide relative to a reaction gas at a temperature of 820° C. Moreover, 1 hour of catalytic reaction process and 3 hours of catalyst regeneration process were alternately conducted.

Since the catalyst used in the method for manufacturing aromatic compounds according to Example 1 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted.

The catalyst prepared by carrying molybdenum on HZSM5 was charged into a reaction column of a fixed-bed flow reactor, the reaction column being formed of Inconel 800H whose portion to be brought into contact with gas is subjected to a calorizing treatment. Upon setting the reaction temperature and the pressure of the interior of the reaction column at 820° C. and 0.15 MPa, respectively, methane to which 3.0% carbon monoxide was added relative to the reaction gas was supplied thereinto at a flow rate or a space velocity (SV) of 3000 ml/hr/g-MFI, thereby conducting an aromatization reaction of methane (the catalytic reaction process). The reaction process was conducted for. 1 hour. After the reaction process, hydrogen gas serving as a regeneration gas was supplied at a flow rate or a space velocity (SV) of 3000 ml/hr/g-MFI upon setting the reaction temperature and the pressure of the interior of the reaction column at 820° C. and 0.15 MPa, respectively, thereby conducting a reaction for regenerating the catalyst (the catalyst regeneration process). The catalyst regeneration process was conducted for 3 hours. Then, an evaluation of the catalytic activity in a reaction for forming benzene and hydrogen by the aromatization reaction of methane was performed.

### Comparative Example 4

In Comparative Example 4, the method for manufacturing aromatic compounds according to the present invention was embodied by initiating a contact reaction between a catalyst and pure methane in such a manner as to initiate a catalytic reaction at a temperature of 820° C., thereby producing an aromatic compound and hydrogen gas. Additionally, 1 hour of catalytic reaction process and 3 hours of catalyst regeneration process were alternately conducted.

Since the catalyst used in the method for manufacturing aromatic compounds according to Comparative Example 4 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of a reaction gas were all the same to those in Example 1, so that a detailed explanation therefor is omitted too.

### Comparative Example 5

In Comparative Example 5, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 1.5% carbon dioxide relative to a reaction gas at a temperature of 820° C. Additionally, 1 hour of catalytic reaction process and 3 hours of catalyst regeneration process were alternately conducted.

Since the catalyst used in the method for manufacturing aromatic compounds according to Comparative Example 5 of the present invention was the same to the catalyst (Mo-HZSM5) used in Referential Example 1, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of an additive gas were all the same to those in Example 1, so that a detailed explanation therefor is omitted too.

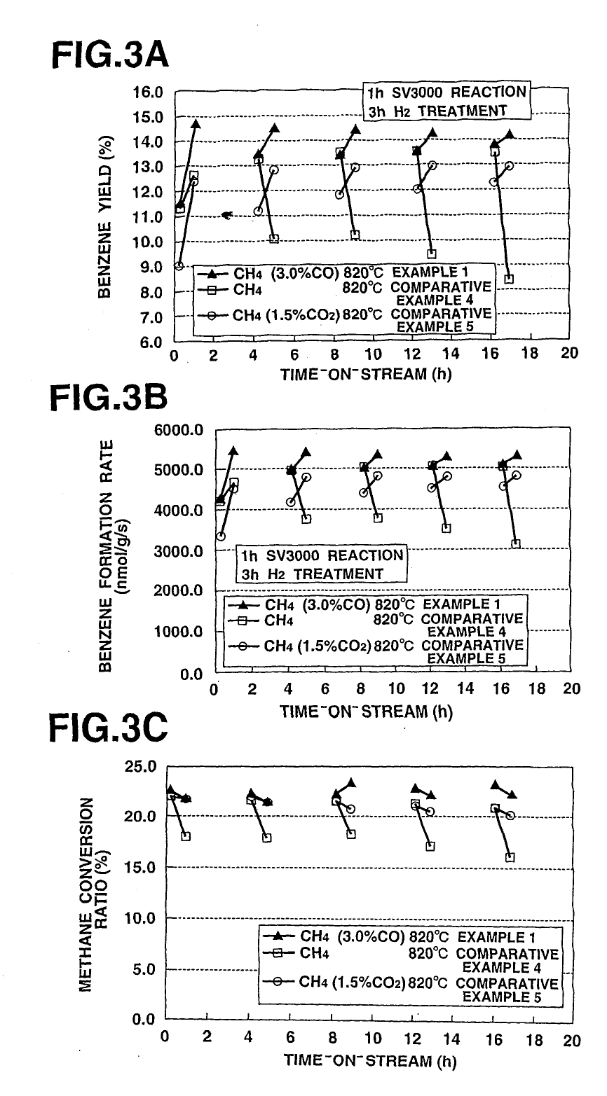

FIG. 3 is a graph showing changes in benzene yield with time, obtained by repeating a catalytic reaction process and a catalyst regeneration process in the presence of a Mo-HZSM5 catalyst under reaction conditions discussed in Example 1 and Comparative Examples 4 and 5.

The case where the carbon monoxide-added methane was used for the contact reaction (Example 1) was not only prevented from degradation in catalytic activity but also improved in maximal benzene yield as compared with the case where pure methane was used in the contact reaction (Comparative Example 4). The case where the carbon dioxide-added methane was used for the contact reaction (Comparative Example 5) was also prevented from degradation in catalytic activity as compared with the case where pure methane was used in the contact reaction (Comparative Example 4), but the maximal benzene yield of Comparative Example 5 never exceeded that in the case where pure methane was used in the contact reaction (Comparative Example 4), as shown in FIG. 3A. The case where the carbon monoxide-added methane was used for the contact reaction (Example 1) was improved in methane conversion ratio as compared with Comparative Examples 4 and 5 thereby being improved also in benzene yield, as shown in FIG. 3C.

(4) Changes in Catalytic Activity With the Addition of Carbon Monoxide, Caused by the Difference in Reaction Temperature

### Example 2

In Example 2, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 3.0% carbon monoxide relative to a reaction gas at a temperature of 890° C.

The catalyst used in the method for manufacturing aromatic compounds according to Example 2 of the present invention was the same to the catalyst (Mo-HZSM5) used in Referential Example 1, with the exception that the catalyst was in the form of a fine powder, so that a detailed explanation of a preparation method therefor is omitted. In other words, the catalyst used in Example 2 was a fine powdery one prepared by impregnating HZSM5 with molybdenum to carry it, drying the Mo-carried substrate and then calcining the obtained catalyst powder. Furthermore, a pretreatment for the catalyst, and also analysis methods for respective substances were the same to those in Referential Example 1, so that detailed explanations therefor are omitted.

The catalyst (0.4 g) prepared by carrying molybdenum on HZSM5 was charged into a reaction column of a fixed-bed flow reactor, the reaction column being formed of glass. Upon setting the reaction temperature and the pressure of the interior of the reaction column at 890° C. and 0.15 MPa, respectively, methane to which 3.0% carbon monoxide was added relative to the reaction gas was supplied thereinto at a flow rate or a space velocity (SV) of 10000 ml/hr/g-MFI, thereby conducting an aromatization reaction of methane.

Then, an evaluation of the catalytic activity in a reaction for forming benzene and hydrogen by the aromatization reaction of methane was performed.

### Example 3

In Example 3, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 3.0% carbon monoxide relative to a reaction gas at a temperature of 870° C.

Since the catalyst used in the method for manufacturing aromatic compounds according to Example 3 of the present invention was the same to the catalyst (Mo-HZSM5) used in Example 2, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of the temperature were all the same to those in Example 2, so that a detailed explanation therefor is omitted too.

### Example 4

In Example 4, the method for manufacturing aromatic compounds according to the present invention was embodied in a method of producing an aromatic compound and hydrogen gas by initiating a contact reaction between a catalyst and methane, in such a manner as to initiate a catalytic reaction with the addition of 3.0% carbon monoxide relative to a reaction gas at a temperature of 850° C.

Since the catalyst used in the method for manufacturing aromatic compounds according to Example 4 of the present invention was the same to the catalyst (Mo-HZSM5) used in Example 2, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of the temperature were all the same to those in Example 2, so that a detailed explanation therefor is omitted too.

### Comparative Example 6

In Comparative Example 6, the method for manufacturing aromatic compounds according to the present invention was embodied by initiating a contact reaction between a catalyst and pure methane, in such a manner as to initiate a catalytic reaction at a temperature of 890° C. thereby producing an aromatic compound and hydrogen gas.

Since the catalyst used in the method for manufacturing aromatic compounds according to Comparative Example 6 of the present invention was the same to the catalyst (Mo-HZSM5) used in Example 2, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of a reaction gas were all the same to those in Example 2, so that a detailed explanation therefor is omitted too.

### Comparative Example 7

In Comparative Example 7, the method for manufacturing aromatic compounds according to the present invention was embodied by initiating a contact reaction between a catalyst and pure methane, in such a manner as to initiate a catalytic reaction at a temperature of 870° C. thereby producing an aromatic compound and hydrogen gas.

Since the catalyst used in the method for manufacturing aromatic compounds according to Comparative Example 7 of the present invention was the same to the catalyst (Mo-HZSM5) used in Example 2, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of a reaction gas were all the same to those in Example 3, so that a detailed explanation therefor is omitted too.

### Comparative Example 8

In Comparative Example 8, the method for manufacturing aromatic compounds according to the present invention was embodied by initiating a contact reaction between a catalyst and pure methane, in such a manner as to initiate a catalytic reaction at a temperature of 850° C. thereby producing an aromatic compound and hydrogen gas.

Since the catalyst used in the method for manufacturing aromatic compounds according to Comparative Example 8 of the present invention was the same to the catalyst (Mo-HZSM5) used in Example 2, a detailed explanation of a preparation method therefor is omitted. In addition to this, a pretreatment for the catalyst, and also analysis methods for respective substances were the same to those in Referential Example 1, so that detailed explanations therefor are omitted. Furthermore, reaction conditions with the exception of a reaction gas were all the same to those in Example 4, so that a detailed explanation therefor is omitted too.

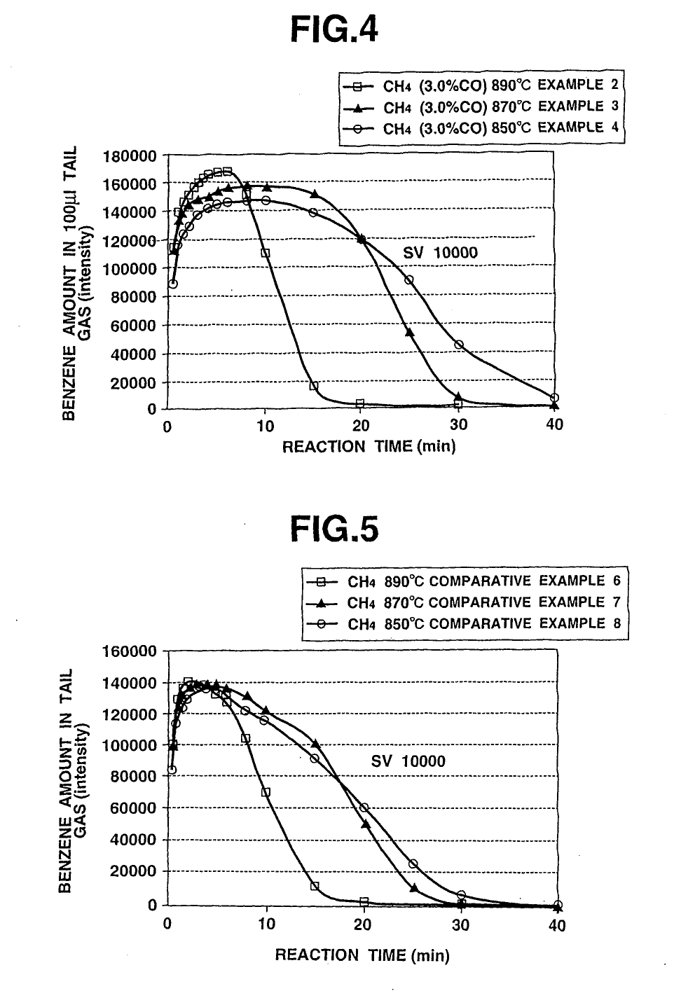

FIG. 4 is a graph showing changes with time in benzene amount in a gas after the reaction, obtained by continuously conducting a catalytic reaction under conditions discussed in Examples 2 to 4 in the presence of a Mo-HZSM5 catalyst. On the other hand, FIG. 5 is a graph showing changes with time in benzene amount in a gas after the reaction, obtained by continuously conducting a catalytic reaction under conditions discussed in Comparative Examples 6 to 8 in the presence of a Mo-HZSM5 catalyst.

As shown in FIG. 4, it is apparent that the case of initiating the contact reaction between the carbon monoxide-added methane and the catalyst was increased in maximal benzene amount in the gas after the reaction, with an increase of the reaction temperature.

On the other hand, the case of initiating the contact reaction between pure methane and the catalyst brought about a little change of the maximal benzene yield in the gas after the reaction.

As discussed above by reference to Examples, it is possible not only to inhibit the catalytic activity degradation but also to improve the maximal benzene yield, by conducting the contact reaction in such a manner as to add carbon monoxide to the reaction gas to be provided to the contact reaction at a reaction temperature of higher than 800° C.

With the addition of carbon monoxide to the reaction gas, the maximal benzene yield is improved with an increase in reaction temperature; therefore, this is particularly effective in the case of requiring a reaction to be instantaneously high in activity, for example; in a reaction using the fluidized-bed reactor.

Additionally, the effect of improving the maximal benzene yield is remarkable at a high SV (i.e., under a condition where the space velocity is high). It is therefore preferable to initiate the contact reaction at an SV of not lower than 3000 ml/hr/g-MFI, particularly at an SV of not lower than 5000 ml/hr/g-MFI.

The difference in a reaction for forming an aromatic compound, between the case of adding carbon monoxide to the gas used in the catalytic reaction and the case of adding carbon dioxide to the same, will be hereinafter discussed.

A reaction for producing benzene (C₆H₆) and hydrogen (H₂) from methane (CH₄) is represented by an equation [1].

6CH₄→C₆H₆+9H₂ [1]

Moreover, a reaction for producing cokes (C) is considered to be a reaction represented by an equation [2].

CH₄→C+2H₂ [2]

Furthermore, a reaction for producing methane may be caused by a reversible reaction of the equation as represented by an equation [3].

C₆H₆+9H₂→6CH₄ [3]

It is considered that a coke-removing reaction is to occur when adding carbon dioxide (CO₂) to the reaction gas.

CO₂+C→2CO [4]

Additionally, it is considered that carbon dioxide is reacted with methane thereby producing hydrogen, as represented by an equation [5].

CO₂+CH₄→2CO+2H₂ [5]

In the case of adding carbon dioxide to the reaction gas, therefore, hydrogen is to be produced by the reaction represented by the equation [5]. Benzene formation is considered to be prevented because a benzene formation reaction represented by the equation [1] is an equilibrium reaction so that the equilibrium is shifted due to the produced hydrogen.

Moreover, carbon dioxide sometimes reduces an active species molybdenum carbide (MoC) by being reacted with molybdenum carbide, as shown in an equation [6]. This reaction is considered to tend to occur at a high flow rate (for example, at a space velocity of 10000 ml/hr/g-MFI).

4CO₂+MoC→MoO₃+5CO [6]

Meanwhile, when adding carbon monoxide (CO), a reversible reaction of the equation [5] is initiated thereby producing carbon dioxide. This carbon dioxide is considered to induce the coke-removing reaction as represented by the equation [4] thereby inhibiting degradation in catalytic activity. It is further considered that hydrogen is consumed in the reversible reaction of the equation [5] thereby shifting the equilibrium so as to accelerate the benzene formation.

Moreover, carbon monoxide is considered to cause a reaction represented by an equation [7]. This reaction also is an equilibrium reaction.

CO→C+O [7]

It is considered that an oxygen atom formed by the equation [7] consumes hydrogen in the reactor thereby shifting the equilibrium of the equation [1] so as to accelerate the benzene formation.

As discussed above, the method for manufacturing aromatic compounds and hydrogen in the use of a catalyst for aromatizing lower hydrocarbons, according to the present invention can produce aromatic hydrocarbons such as benzene and the like with high yields. More specifically, it becomes possible not only to maintain the catalytic activity over a long period of time but also to obtain a yield sufficient for practical use, with a reaction temperature of higher than 800° C. and with the addition of carbon monoxide.

In other words, it becomes possible not only to inhibit the accumulation of the hard-to-remove cokes by adding carbon monoxide, but also to obtain a maximal benzene yield of not less than that obtained in the case of adding methane only by increasing the temperature of the catalytic reaction. On the contrary, CO₂ has the effect of inhibiting the aromatization reaction so as not able to obtain a benzene yield (or a catalytic activity) of not smaller than the maximal benzene yield of the case of adding methane only.

Particularly in the process of repeating the catalytic reaction process and the catalyst regeneration process, the yield at the initial stage of the reaction is important. In the process for producing aromatic hydrocarbons, according to the present invention, it is possible not only to obtain benzene at high yields but also to inhibit the formation of deposited cokes hard to remove for regeneration. With this, high catalytic activities are maintained over a long period of time even after repeating the catalytic reaction and the catalyst regeneration reaction.

Although the invention has been described above by reference to certain embodiments and examples of the invention, the invention is not limited to the embodiments and examples described above. Modifications and variations of the embodiments and examples described above will occur to those skilled in the art, in light of the above teachings.

The present invention is characterized by accelerating the removal of cokes with the addition of carbon monoxide to the reaction gas containing lower hydrocarbons and by improving the benzene formation rate. The catalyst for aromatizing lower hydrocarbons is, therefore, not limited to molybdenum carried on metallosilicate; more specifically, it is apparent that effects equal to those in the Mo-carried catalyst can be obtained also when rhenium, tungsten, iron, cobalt and a compound of these (including molybdenum), selected from various catalytic metals whose effect as the catalyst for aromatizing lower hydrocarbons has already been confirmed and discussed in some reference documents (e.g., “Surface”, Vol. 37, No. 12 (1999), pages 71-81, “Catalytic chemical conversion of methane-Direct synthesis of benzene using molded zeolite catalyst”), are used singly or in combination.

Additionally, in the case of repeating the catalytic reaction process and the catalyst regeneration process, a reaction time and a regeneration time are not limited to the examples and therefore may be suitably determined within a time period of not depositing the hard-to-remove cokes, for example, by switching from the catalytic reaction process to the catalyst regeneration process before the catalytic activity is reduced, on the basis of changes in catalytic activity. For instance, the catalytic reaction process and the catalyst regeneration process may be switched depending on changes in catalyst temperature measured during the catalytic reaction process. The reaction for aromatizing lower hydrocarbons is an endothermic reaction, so that the temperature of the catalyst is reduced during the catalytic reaction process. Additionally, the activity in the aromatization reaction of lower hydrocarbons is to decrease with catalyst degradation, so that the degree of catalyst degradation may be detected by measuring changes in catalyst temperature. It is therefore also acceptable to switch from the reaction process to the regeneration process after the catalyst temperature starts increasing, with which aromatic hydrocarbons become more efficiently produced and additionally the catalyst degradation becomes more efficiently prevented. Furthermore, it is possible to save energy for increasing the catalyst temperature up to a temperature required for the reaction in the catalyst regeneration process, by switching into the catalyst regeneration process after the catalyst temperature is increased.

Moreover, in the case of repeating the reaction process and the regeneration process, the regeneration gas used in the regeneration process is not limited to hydrogen, and suitably usable if containing a reducing gas such as carbon monoxide and the like.

1. A method for manufacturing aromatic compounds which method produces hydrogen and aromatic compounds containing an aromatic hydrocarbon as the principal component by initiating a contact reaction between a lower hydrocarbon and a catalyst, characterized in that:
carbon monoxide is added to the lower hydrocarbon; and
the contact reaction is initiated between the lower hydrocarbon and the catalyst at a reaction temperature of higher than 800° C.

2. A method for manufacturing aromatic compounds, as claimed in claim 1, characterized in that the carbon monoxide has a concentration of from 0.75 to 20% relative to a reaction gas.

3. A method for manufacturing aromatic compounds, as claimed in claim 1, characterized in that the reaction temperature is not lower than 820° C.

4. A method for manufacturing aromatic compounds, as claimed in claim 1, characterized in that:
the aromatic compounds are produced by repeating a reaction process of initiating the contact reaction between the lower hydrocarbon and the catalyst and a regeneration process of regenerating the catalyst used in the reaction process.
